(19) United States
(12) Patent Application Publication
(10) Pub. No.: US 2009/0248021 A1
(43) Pub. Date: Oct. 1, 2009

(54) End Effector Assembly for Electrosurgical Devices and System for Using the Same

(75) Inventor: Nicole McKenna, Boulder, CO (US)

Correspondence Address: Tyco Healthcare Group LP, 60 MIDDLETOWN AVENUE, NORTH HAVEN, CT, 06473, US

(73) Assignee: TYCO Healthcare Group LP

(21) Appl. No.: 12/400,901

(22) Filed: Mar. 10, 2009

Related U.S. Application Data
(60) Provisional application No. 61/041,065, filed on Mar. 31, 2008.

(51) Int. Cl. A61B 18/04

(52) U.S. Cl. 606/51

(57) ABSTRACT

A bipolar forceps is provided and includes a housing having one or more shafts that extend therefrom that operatively support an end effector assembly at a distal end thereof. The end effector assembly includes first and second jaw members. A tissue sealing plate disposed on each of the jaw members is provided. The tissue sealing plates is configured to support a plurality of electrodes thereon and arranged in vertically opposing pairs along the length of the jaw members. The plurality of electrodes is adapted to independently connect to an electrosurgical energy source such that each vertically opposing electrode pairs form an independently controllable electrical circuit when tissue is held between the jaw members. A control system having one or more algorithms for independently controlling and/or monitoring the delivery of electrosurgical energy from the electrosurgical energy source to the plurality of electrodes is also provided.

### CROSS-REFERENCE TO RELATED APPLICATIONS

This application claims the benefit of priority to U.S. Provisional Application Ser. No. 61/041,065 entitled “END EFFECTOR ASSEMBLY FOR ELECTROSURGICAL DEVICES AND SYSTEM FOR USING THE SAME,” filed Mar. 31, 2008 by Nicole McKenna, which is incorporated by reference herein.

### BACKGROUND

1. Technical Field

The present disclosure relates to an electrosurgical forceps and, more particularly, the present disclosure relates to electrosurgical forceps, for use with either an endoscopic or open electrosurgical forceps for sealing, cutting, and/or coagulating tissue, which employ opposing jaw members each having seal plates including selectively independently controllable electrodes.

2. Description of Related Art

Electrosurgical forceps utilize both mechanical clamping action and electrical energy to effect hemostasis by heating the tissue and blood vessels to coagulate, cauterize and/or seal tissue.

By utilizing an endoscopic electrosurgical forceps, a surgeon can cauterize, coagulate/desiccate, seal, and/or simply reduce or slow bleeding simply by controlling the intensity, frequency and duration of the electrosurgical energy applied through the jaw members to the tissue. Most small blood vessels, i.e., in the range below two millimeters in diameter, can often be closed using standard electrosurgical instruments and techniques. However, if a larger vessel is ligated, it may be necessary for the surgeon to convert the endoscopic procedure into an open-surgical procedure and thereby abandon the benefits of endoscopic surgery. Alternatively, the surgeon can seal the larger vessel or tissue.

It is thought that the process of coagulating vessels is fundamentally different than electrosurgical vessel sealing. For the purposes herein, “coagulation” is defined as a process of desiccating tissue wherein the tissue cells are ruptured and dried. “Vessel sealing” or “tissue sealing” is defined as the process of liquefying the collagen in the tissue so that it reforms into a fused mass. Coagulation of small vessels is sufficient to permanently close them, while larger vessels need to be sealed to assure permanent closure.

As mentioned above, electrosurgical forceps utilize electrosurgical energy to effect hemostasis by heating the tissue and vessels to coagulate, cauterize and/or seal tissue. The source of electrosurgical energy is configured to provide a voltage across tissue, which, in turn, causes current to flow therethrough. The current passing through the tissue, which is acting as a resistor, causes electrical power to be delivered to that portion of tissue (P=I²*R), resulting in a transfer of energy to the tissue in the form of heat.

Typically, the source of electrosurgical energy will be in operative communication with a computer that includes a control algorithm configured to monitor, measure, and/or control the amount of electrosurgical energy that is being delivered to the vessel sealing site. The computer and control algorithm are usually configured to monitor and measure one or more electrical parameters at the tissue sealing site. Common in the art is to have the computer and/or control algorithm configured to measure impedance at the tissue sealing site. When a threshold value of impedance is detected by the computer and/or control algorithm, the electrosurgical energy being transmitted to the tissue sealing site is adjusted accordingly. The measured impedance may be detected, measured, and transmitted to the control algorithm via sensors located on the electrosurgical forceps and in operative communication the computer.

Electrosurgical forceps also include end effector assemblies that include opposing jaw members pivotally connected to each other and each having a seal plate configured to cause a tissue effect when tissue is grasped therebetween. The seal plates are employed to transmit electrosurgical energy to the tissue sealing site when the jaw members are in a closed configuration. Each seal plate typically extends the length of their respective jaw member, or portion thereof. The seal plates may be in operative communication with the control algorithm via the sensors.

During vessel sealing procedures, in some instances, eschar may form and accumulate on the seal plates (e.g., proximal end of one or both of the seal plates). As is known in the art, because eschar is highly resistive it tends to act like a resistor and impedes the flow current. As a result, the impedance measured across tissue, at the vessel sealing site, may be inaccurate for purposes of controlling the amount of electrosurgical energy delivered to the tissue sealing site. That is, because the total impedance measured at the tissue sealing site is now a combination of both the resistance of the tissue and the resistance of the eschar formed on one or both of the seal plates, the measured impedance may not be an entirely accurate representation of the actual impedance of the tissue as the tissue is being cooked. Consequently, and as will be discussed in greater detail below, non-uniform and/or incomplete tissue seals may form when eschar builds on tissue sealing plates. This anomaly becomes of particular concern in instances where the jaw members have been designed to have a longer length to accommodate certain tissue types.

### SUMMARY

A bipolar forceps is provided. The bipolar forceps includes a housing having one or more shafts which extends therefrom that operatively supports an end effector assembly at a distal end thereof The end effector assembly includes first and second jaw members pivotably connected to each other and moveable from an open spaced apart position to a closed position to grasp tissue. The bipolar forceps also includes a tissue sealing plate disposed on each of the jaw members, wherein each tissue sealing plate is configured to support a plurality of electrodes thereon arranged in vertically opposing pairs along the length of the jaw members. Each of the plurality of electrodes is adapted to independently connect to an electrosurgical energy source such that each vertically opposing electrode pair forms an independently controllable electrical circuit when tissue is held between the first and second jaw members. A control system having one or more algorithms for independently controlling and/or monitoring the delivery of electrosurgical energy from the electrosurgical energy source to the plurality of electrodes is also provided.

The one or more algorithms are configured to determine a threshold condition when an electrical parameter is reached. The electrical parameter is selected from the group consisting of impedance, voltage, or current. Moreover, the one or more algorithms are configured to re-route the electrosurgical energy to only those electrode pairs which require additional electrosurgical energy to reach an end seal condition when the threshold condition is reached. In operation, an abnormal electrode pair “end seal” condition causes the one or more algorithms to execute an override command if certain other electrical or physical conditions do not correlate to a safe end seal condition. Alternatively, an abnormal electrode pair “end seal” condition causes the one or more algorithms to execute a re-grasp alarm if certain other electrical or physical conditions do not correlate to a safe end seal condition.

In embodiments the plurality of electrodes is insulated from each other by a non-conductive material.

The control system is configured to query the electrode pairs individually and/or in together upon a condition being met prior to adjusting electrical delivery. A bipolar forceps according to claim 1, wherein the control system

A method for performing an electrosurgical procedure is also provided including the initial step of providing a bipolar forceps. The bipolar forceps includes a housing having one or more shafts which extends therefrom that operatively supports an end effector assembly at a distal end thereof. The end effector assembly includes first and second jaw members pivotably connected to each other and moveable from an open spaced apart position to a closed position to grasp tissue. The bipolar forceps includes a tissue sealing plate disposed on each of the jaw members, wherein each tissue sealing plate is configured to support a plurality of electrodes thereon arranged in vertically opposing pairs along the length of the jaw members. Each of the plurality of electrodes is adapted to independently connect to an electrosurgical energy source such that each vertically opposing electrode pair forms an independently controllable electrical circuit when tissue is held between the first and second jaw members. The bipolar forceps includes a control system having one or more algorithms for independently controlling and/or monitoring the delivery of electrosurgical energy from the electrosurgical energy source to the plurality of electrodes. The method for performing an electrosurgical procedure also includes the steps of: delivering electrosurgical energy from the source of electrosurgical energy to the plurality of electrodes on each of the seal plates; measuring the impedance levels across tissue at each of the plurality of electrodes; comparing the measured values of impedance levels at each of the plurality of electrodes with known threshold values of impedance; and adjusting the amount of electrosurgical energy being delivered to each of the plurality of electrodes as needed.

### BRIEF DESCRIPTION OF THE DRAWING

FIG. 1 is a perspective view of a bipolar forceps in accordance with the present disclosure;

FIG. 2 illustrates an electrical wiring diagram for the bipolar forceps depicted in FIG. 1 in accordance with the present disclosure;

FIG. 3 is s side cross-sectional view of jaw members in accordance with the present disclosure;

FIG. 4 is an exploded with of the jaw member depicted in FIG. 3; and

FIG. 5 is a flow chart illustrating a method for performing an electrosurgical procedure in accordance with the present disclosure.

### DETAILED DESCRIPTION

Detailed embodiments of the present disclosure are disclosed herein; however, it is to be understood that the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

During electrocautery surgical procedures such as sealing it is common for eschar to form and accumulate on the seal plates, or portions thereof. Typically, eschar develops at or near a proximal end of the seal plate; this area of the seal plate is commonly referred to in the art, and hereinafter referred to as the “heel” of the seal plate. As the amount of eschar forming and accumulating near the heel of the seal plates increases, so too does the impedance at the tissue sealing site; this is because eschar impedes current flow. As described above, this is especially evident if the seal plates are longer than the potential heat transfer.

As a result of the formation and accumulation of eschar on the seal plates, inaccurate impedance measurements may be communicated to the control algorithm in the generator. Subsequently, these inaccurate impedance measurements are implemented by the control algorithm in calculating and determining whether a threshold level of impedance has been reached which may result in any number of possible errors. For example, if the resultant threshold impedance levels calculated by the control algorithm are in all actuality a false representation of the actual impedance present at the tissue sealing site, the control algorithm is eventually “tricked” into causing the source of electrosurgical energy to be adjusted prematurely, which can ultimately lead to ineffective tissue seals being formed, which may lead to leakage at or near the proximal thrombosis.

Having end effector assemblies, as described herein, which include seal plates defining multiple electrodes that are independently monitored and controlled greatly reduces the chances of false impedance measurements being transferred to the generator and, as a result reduces the likelihood of a weak or ineffective seal being passed off as “complete”.

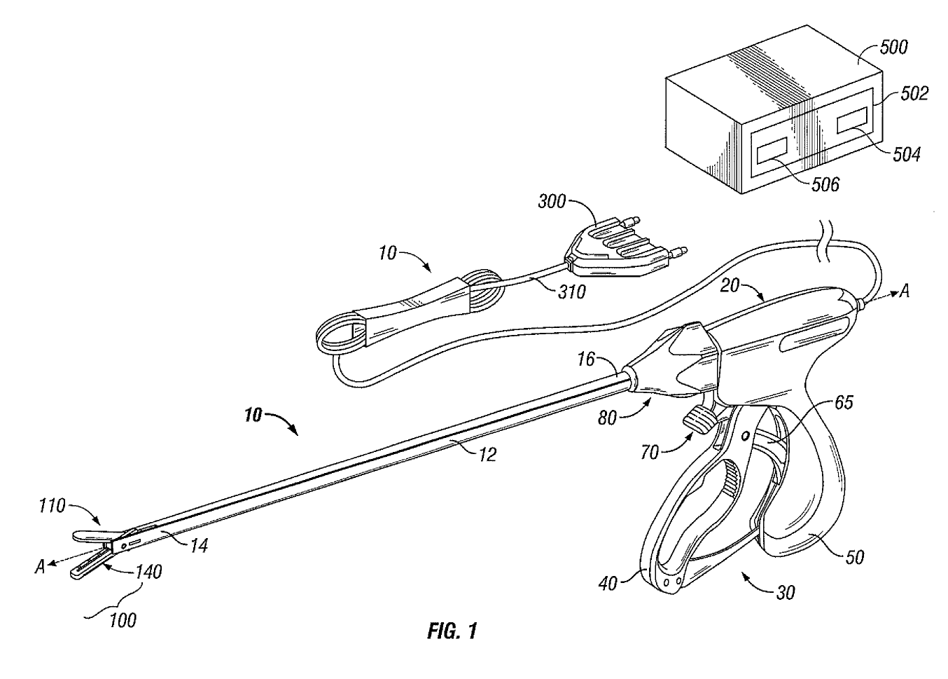

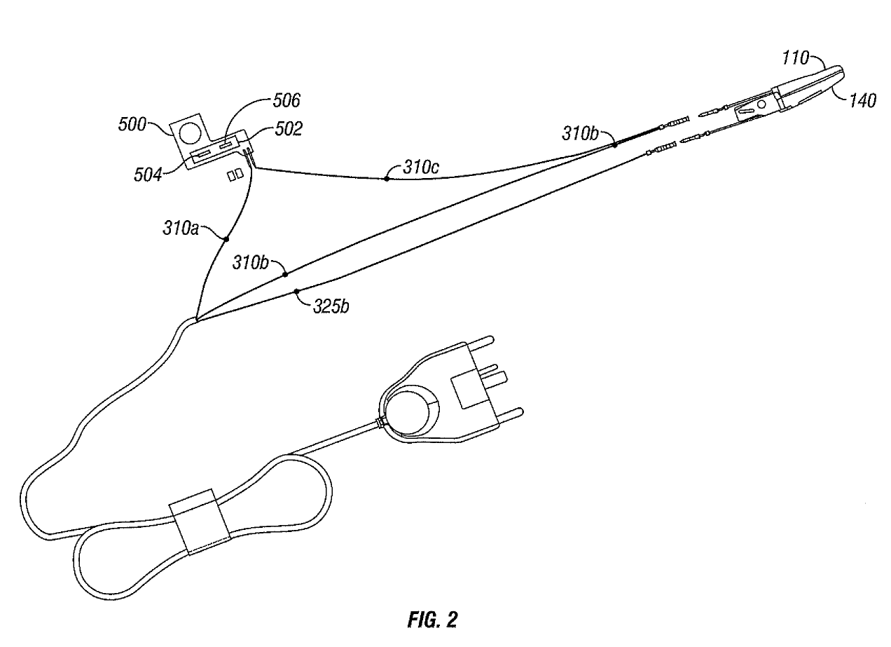

Turning now to FIG. 1 one embodiment of an electrosurgical forceps 10 in accordance with the present disclosure is shown. For the remainder of the disclosure it will be assumed that the electrosurgical forceps is an endoscopic bipolar forceps, as seen in FIG. 1, keeping in mind that any electrosurgical forceps may be employed with the present disclosure. For example, although the majority of the figure drawings depict a bipolar forceps 10 for use in connection with endoscopic surgical procedures, the present disclosure may be used for more traditional open surgical procedures. For the purposes herein, the forceps 10 is described in terms of an endoscopic instrument; however, it is contemplated that an open version of the forceps may also include the same or similar operating components and features as described below.

Bipolar forceps 10 is shown for use with various electrosurgical procedures and generally includes a shaft 12, a housing 20, a handle assembly 30, a rotating assembly 80, a trigger assembly 70, and an end effector assembly 100, which is operatively connected to a drive assembly. End effector assembly 100 includes opposing jaw members 110 and 140, which mutually cooperate to grasp, seal and, in some cases, divide large tubular vessels and large vascular tissues.

Shaft 12 includes a distal end 14 that mechanically engages the end effector assembly 100 and a proximal end 116 that mechanically engages the housing 20. In the drawings and in the descriptions which follow, the term “proximal,” as is traditional, will refer to the end of the forceps 10 which is closer to the user, while the term “distal” will refer to the end which is farther from the user.

Handle assembly 30 includes a fixed handle 50 and a movable handle 40. Fixed handle 50 is integrally associated with housing 20 and handle 40 is movable relative to fixed handle 50. Fixed handle 50 may include one or more ergonomic enhancing elements to facilitate handling, e.g., scallops, protuberances, elastomeric material, etc.

Movable handle 40 of handle assembly 30 is ultimately connected to the drive assembly which together mechanically cooperate to impart movement to the jaw members 110 and 140 to move from an open position, wherein the jaw members 110 and 140 are disposed in spaced relation relative to one another, to a clamping or closed position, wherein the jaw members 110 and 140 cooperate to grasp tissue therebetween.

Rotating assembly 80 is operatively associated with the housing 20 and is rotatable approximately 180 degrees about a longitudinal axis “A-A” defined through shaft 12 (see FIG. 1).

Forceps 10 also includes an electrosurgical cable 310 which connects the forceps 10 to a source of electrosurgical energy, e.g., a generator 500 (shown schematically). It is contemplated that generators such as those sold by Valleylab—a division of Tyco Healthcare LP, located in Boulder Colorado may be used as a source of electrosurgical energy, e.g., Ligasure™ Generator, FORCE EZ™ Electrosurgical Generator, FORCE FX™ Electrosurgical Generator, FORCE 1C™, FORCE 2™ Generator, SurgiStat™ II or other envisioned generators which may perform different or enhanced functions.

Cable 310 is internally divided into cable leads 310a, 310b and 325b which are designed to transmit electrical potentials through their respective feed paths through the forceps 10 to the end effector assembly 100. More particularly, cable feed 325b connects through the forceps housing 20 and through the rotating assembly to jaw member 120. Lead 310a connects to one side of the switch 60 and lead 310c connects to the opposite side of the switch 60 such that upon activation of the switch energy is transmitted from lead 310a to 310c. Lead 310c is spliced with lead 310b which connects through the rotating assembly to jaw member 110.

For a more detailed description of shaft 12, handle assembly 30, movable handle 40, rotating assembly 80, electrosurgical cable 310 (including line-feed configurations and/or connections), and the drive assembly reference is made to commonly owned patent application Ser. No. 10/369,894, filed on Feb. 20, 2003, entitled VESSEL SEALER AND DIVIDER AND METHOD OF MANUFACTURING THE SAME.

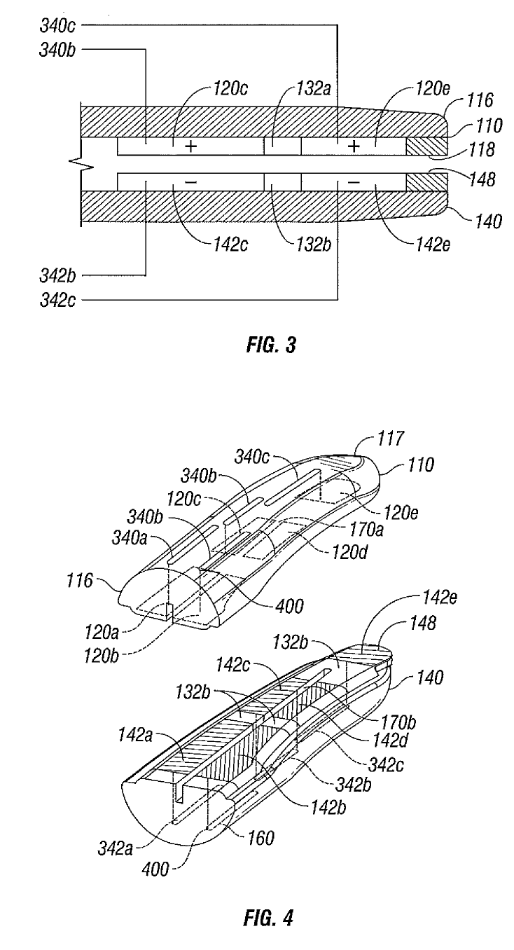

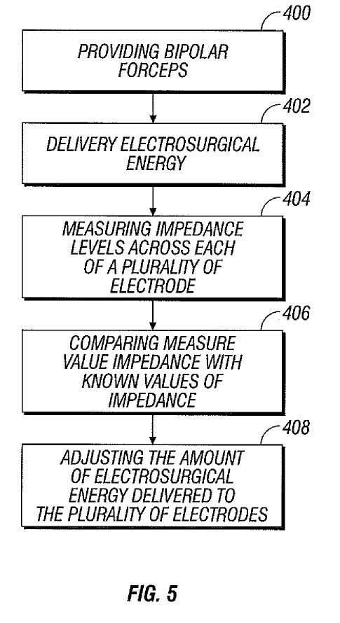

As shown in FIGS. 3 and 4, end effector assembly 100 includes opposing jaw members 110 and 140. Jaw members 110 and 140 are generally symmetrical and include similar component features which cooperate to effectively sealing and divide tissue. As a result and unless otherwise noted, only jaw member 110 and the operative features associated therewith are described in detail herein but as can be appreciated, many of these features apply to jaw member 140 as well.

Jaw member 110 includes an insulative structural substrate or support member 116 and an electrically conductive tissue sealing plate 118 (hereinafter seal plate 118). The insulator 116 may be dimensioned to securely engage the seal plate 118 by stamping, by overmolding, by metal injection or other known manufacturing techniques. All of these manufacturing techniques produce an electrode having a seal plate 118 which is substantially surrounded by insulating substrate 116. Jaw member 140 includes a structural support member 146 and an electrically conducive seal plate 148.

With continued reference to FIGS. 3 and 4, seal plates 118 and 148 are configured in such a manner that electrical current travels from one seal plate (e.g., 118) through tissue to the opposing seal plate (e.g., 148). More particularly, end effector assembly 100 is configured to include a series of opposing electrode pairs disposed within the tissue sealing surfaces of each jaw member 110 and 140, respectively. More particularly, each jaw member, e.g., 110, includes series of electrode 120a-120e spaced along the tissue sealing surface 118 thereof from the proximal end 116 to a distal end 117 thereof and across the tissue sealing surface 118. The electrodes 120a-120e are spaced relative to one another and are either separated by a knife channel 170a defined in the jaw member 110 (from the proximal end to the distal end of the jaw member 110) or an insulative material 132a disposed therebetween. The electrodes 120a-120e may be arranged in pairs, e.g., 120a and 120b, 120c and 120d or may be randomly arranged along the tissue surface depending upon a particular purpose.

Jaw member 140 includes a series of corresponding electrodes 142a-142e which oppose respective electrodes 120a-120e disposed on jaw member 110 such that during activation each pair of opposing electrodes, e.g., 120a and 142a, 120b and 142b, 120c and 142c, 120d and 142d, and 120e and 142e treat tissue disposed therebetween to form a tissue seal. Much like electrodes 120a-120e, electrodes 142a-142e are spaced relative to one another by a knife channel 170b or an insulative material 132b disposed therebetween. As explained in more detail below, each electrode pair, e.g., 120a and 142a, is controlled and monitored by a computer 504 operatively coupled to generator 500.

Seal plates 118 and 148 may be configured in such a manner that when jaw members 110 and 140 are in a closed configuration, a knife blade, not shown, or portion thereof, may translate within channels 170a and 170b defined by seal plates 118 and 148.

Generator 500 is configured to control and/or monitor one or more electrode pairs operatively coupled to or disposed on seal plates 118 and 148 of jaw members 110 and 140, respectively. Generator 500 includes a control system 502 having one or more computers and/or computer programs 504 which include one or more control algorithms.

Computer 504 is housed within electrosurgical generator 500 and disposed in operative communication therewith via the community circuitry, not shown, associated with generator 500. One or more controls 506 may be utilized to set certain parameters of the computer 504. Within the purview of the present disclosure, computer 504 may be remotely located with respect to generator 500. Here, generator 500 and computer 504 may be operatively connected to each other via any number of wire or wireless connections known in the art.

Computer 504 include any number of computer programs, software modules and drivers associated therewith such that electrosurgical generator 500 functions to control or monitor each individual electrode to enhance the sealing procedure. For example, computer 504 captures and receives input data from the electrodes, either individually (or in pairs across the width of the jaw surface), and transmits the captured and received input data to an input algorithm of the computer 504. The input data is representative of one or more electrical parameters associated with electrosurgical sealing, (e.g., tissue impedance). Based on the input data received, computer 504 controls the amount of electrosurgical energy to each electrode.

As mentioned above, the internal control algorithm(s) of computer 504 are configured to receive the input data and execute internal code to compare impedance levels measured along the tissue sealing surfaces 118, 148 proximate each individual opposing electrode pair, e.g., opposing electrode pair 120a and 142a, 120b and 142b, 120c and 142c, 120d and 142d, and 120e and 142e, with known threshold levels of impedance stored in, or accessible to the control algorithm. As impedance levels measured at the tissue sealing site approaches and/or reaches known threshold levels of impedance, the control algorithm of computer 504 independently monitors and controls the amount of electrosurgical energy that is delivered to each electrode pair, e.g., electrode pair 120a and 142a, which provides a more accurate seal geometry between opposing tissue sealing surfaces 118 and 148 especially with longer jaw lengths. In other words, each individual sealing site respective to each electrode pair 120a and 142a may be accurately controlled and monitored to deliver the optimum amount of energy to tissue, reduce the chances of eschar buildup and maximize seal quality across the entire tissue sealing surfaces 118 and 148.

The computer 504 (and algorithms disposed therein) may be configured to monitor and control the end or shut off parameters for each respective pair of electrodes 120a and 142a, 120b and 142b, 120c and 142c, 120d and 142d, and 120e and 142e before an “end seal” signal will be reached Energy may be re-routed to only those electrode pairs which require additional energy or longer “cook time” to reach an end seal condition. As mentioned above, an abnormal electrode pair “end seal” condition may be met with some scrutiny by the algorithm and, may be overridden if certain other electrical or physical conditions do not correlate to an end seal condition or a re-grasp alarm may be triggered to avoid an unsafe condition.

As best shown in FIGS. 3 and 4, computer 504 is operatively connected with each electrode pair, e.g., 120a and 142a, via one or more of cable connections 340a-340c and 342a-342c, respectively. Various types of electrical connections may be utilized which are known in the art and the electrical connections may be routed through the instrument shaft(s) and connected to one or more printed circuit boards (PCBs) disposed within the forces 10.

The electrode pairs, e.g., e.g., 120a and 142a, 120b and 142b, 120c and 142c, 120d and 142d, and 120e and 142e are sized, configured and arranged in such a manner that tissue to be sealed contacts a sufficient number of electrodes to optimize seal quality. For example, larger electrodes pairs, e.g., 120a and 142a and 120e and 142e may be positioned at the proximal and distal ends, respectively, of the tissue sealing surfaces 118 and 148 and smaller electrode pairs 120c and 142c may be more centrally disposed on the tissue sealing surfaces to optimize sealing. The electrodes 120a-120e and 142a-142e may be symmetrically or asymmetrically arranged on either side of knife slots 170a and 170b in opposing pairs depending upon a particular purpose of to optimize sealing a particular tissue type.

One or more sensors 400 may be utilized to detect one or more other electrical of physical parameters, e.g., temperature, optical clarity, tissue expansion, rate of tissue expansion, pressure, etc. and relay the information back to the generator 500 for utilization by one or more algorithms of computer 504 to regulate energy delivery. Sensors 400 may include thermocouples, photodiodes, transducers, accelerometers, microsensors manufactured using MEMS technology or other types of sensors are contemplated and within the purview of the present disclosure.

The present disclosure also provides a method for performing an electrosurgical procedure. At step 400, an electrosurgical forceps is provided. At step 402, electrosurgical energy is delivered from the source of electrosurgical energy to the plurality of electrodes on each of the seal plates. At step 404, the impedance level across tissue at each of the plurality of electrodes is measured. At step 406, the measured values of impedance levels at each of the plurality of electrodes are compared with known threshold values of impedance. At step 408, the amount of electrosurgical energy being delivered to each of the plurality of electrodes is adjusted as needed.

From the foregoing and with reference to the various figure drawings, those skilled in the art will appreciate that certain modifications can also be made to the present disclosure without departing from the scope of the same.

For example, while it is shown in the figures that electrodes 120a-120e and 142a-142e of respective seal plates 118 and 148 having the same charge, it is within the purview of the present disclosure that the electrodes or different opposing pairs of electrodes defined by their respective seal plates may have alternating potentials along the seal surfaces. Obviously, in this instance, the electrical connections would have to be slightly altered to accomplish this purpose.

In one embodiment, the computer 504 measures the impedance (or other electrical parameters) in real time and continually adjusts the electrical output in real time to optimize the tissue seal. In another embodiment, the computer 504 is configured to measure the electrical parameters in real time and make adjustments over a pre-set time period, only upon a threshold condition being met or after a pre-set number of pulses. The computer 504 may also be configured to further query the electrode pairs either individually or in pairs upon a condition being met prior to adjusting electrical delivery. For example, the computer 504 may be configured to query one or more adjacent electrode pairs (or one or more sensors) for further electrical information (or other information relating to temperature, pressure, rate of tissue expansion, optical clarity, etc) before altering electrical delivery if an abnormal electrical condition has occurred prior to adjusting the delivery of electrical energy.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A bipolar forceps, comprising:
a housing having at least one shaft that extends therefrom that operatively supports an end effector assembly at a distal end thereof, the end effector assembly including first and second jaw members pivotably connected to each other and moveable relative to one another from an open spaced apart position to a closed position to grasp tissue;
a tissue sealing plate disposed on each of the jaw members, each tissue sealing plate being configured to support a plurality of electrodes thereon arranged in vertically opposing pairs along the length of the jaw members, each of the plurality of electrodes adapted to independently connect to an electrosurgical energy source such that each vertically opposing electrode pair forms an independently controllable electrical circuit when tissue is grasped between the first and second jaw members; and
a control system having at least one algorithm for at least one of independently controlling and monitoring the delivery of electrosurgical energy from the electrosurgical energy source to the plurality of electrodes.

2. A bipolar forceps according to claim 1, wherein the at least one algorithm is configured to determine a threshold condition when an electrical parameter is reached, the electrical parameter selected from the group consisting of impedance, voltage, and current.

3. A bipolar forceps according to claim 1, wherein the plurality of electrodes are insulated from each other by a non-conductive material.

4. A bipolar forceps according to claim 1, wherein the at least one algorithm is configured to re-route the electrosurgical energy to only those electrode pairs that require additional electrosurgical energy to reach an end seal condition when the threshold condition is reached.

5. A bipolar forceps according to claim 1, wherein an abnormal electrode pair “end seal” condition causes the at least one algorithm to execute an override command if certain other electrical or physical conditions do not correlate to a safe end seal condition.

6. A bipolar forceps according to claim 1, wherein an abnormal electrode pair “end seal” condition causes the at least one algorithm to execute a re-grasp alarm if certain other electrical or physical conditions do not correlate to a safe end seal condition.

7. A bipolar forceps according to claim 1, wherein the control system is configured to query the electrode pairs individually upon a condition being met prior to adjusting electrical delivery.

8. A bipolar forceps according to claim 1, wherein the control system is configured to query the electrode pairs together upon a condition being met prior to adjusting electrical delivery.

9. A method for performing an electrosurgical procedure, the method comprising the steps of:
providing a bipolar forceps including first and second jaw members pivotably connected to each other, a tissue sealing plate disposed on each of the jaw members, each tissue sealing plate configured to support a plurality of electrodes thereon arranged in vertically opposing pairs along the length of the jaw members, each of the plurality of electrodes adapted to independently connect to an electrosurgical energy source such that each vertically opposing electrode pair forms an independently controllable electrical circuit when tissue is held between the first and second jaw members;
delivering electrosurgical energy from the electrosurgical energy source to the plurality of electrodes on each of the seal plates;
measuring the impedance levels across tissue at each of the plurality of electrodes;
comparing the measured values of impedance levels at each of the plurality of electrodes with known threshold values of impedance; and
adjusting the amount of electrosurgical energy being delivered to each of the plurality of electrodes as needed.
